(19) United States
(12) Patent Application Publication
(10) Pub. No.: US 2026/0174783 A1
(43) Pub. Date: Jun. 25, 2026

(54) HALOBETASOL PROPIONATE-LIDOCAINE FORMULATIONS

(71) Applicant: Citius Pharmaceuticals, Inc., Cranford, NJ (US)

(72) Inventors: Vijendra Nalamothu, Raleigh, NC (US); Srikanth Manne, Cary, NC (US); Sindhu Addala, Apex, NC (US)

(21) Appl. No.: 19/412,192

(22) Filed: Dec. 8, 2025

Related U.S. Application Data
(63) Continuation of application No. 18/647,921, filed on Apr. 26, 2024.
(60) Provisional application No. 63/498,791, filed on Apr. 27, 2023.
(60) Provisional application No. 63/522,070, filed on Jun. 20, 2023.

(51) Int. Cl. A61K 31/573; A61K 9/00; A61K 9/06; A61K 31/167; A61K 47/10; A61K 47/34; A61K 47/38; A61P 9/14

(52) U.S. Cl. CPC A61K 31/573; A61K 9/0014; A61K 9/06; A61K 31/167; A61K 47/10; A61K 47/34; A61K 47/38; A61P 9/14

(57) ABSTRACT

The disclosure relates to pharmaceutical formulations comprising of lidocaine and halobetasol propionate, methods of making such formulations, and methods of using such formulations in the treatment of hemorrhoids.

### CROSS-REFERENCE TO RELATED APPLICATIONS

The present application is a continuation of U.S. patent application Ser. No. 18/647,921, filed Apr. 26, 2024, which claims the benefit of and priority to U.S. Provisional Application No. 63/498,791, filed Apr. 27, 2023, and U.S. Provisional Application No. 63/522,070, filed Jun. 20, 2023, the entire contents of both of which are incorporated herein by reference.

### TECHNICAL FIELD

The disclosure relates to pharmaceutical formulations comprising lidocaine hydrochloride and halobetasol propionate that are useful in the topical treatment of hemorrhoids.

### BACKGROUND

Symptomatic hemorrhoid disease is the fourth leading outpatient gastrointestinal diagnosis and has a considerable impact on quality of life (Sun et al., Review of hemorrhoid disease: Presentation and management. Clinics in colon and rectal surgery. 2016; 29:22-9). In the United States, hemorrhoids affect nearly 5% of the population, with approximately 10 million patients annually reporting symptoms.

Hemorrhoids are caused by the deterioration of the supporting tissues of the anal cushions, which causes venous dilation and prolapse: factors like advancing age, pregnancy, abdominal obesity, and constipation are thought to increase the risk of hemorrhoid development (Lohsiriwat, Treatment of hemorrhoids: A coloproctologist's view. World journal of gastroenterology. 2015; 21:9245-52). Symptoms include bleeding, tissue prolapse, ulceration, skin irritation, pruritus, and pain (Jacobs, Clinical practice. Hemorrhoids. The New England journal of medicine. 2014; 371:944-51).

Hemorrhoid severity is divided into four grades based on Goligher's classification, the most widely used hemorrhoid categorization system (Goligher, Surgery of anus, rectum and colon: Harcourt Publishers; 1980). Grades I and II represent the large majority of cases and can be controlled with lifestyle changes and medical management, while those with more severe cases such as prolapsed and/or irreducible hemorrhoids often require surgical intervention (Lorenc et al., Tribenoside and lidocaine in the local treatment of hemorrhoids: An overview of clinical evidence. European review for medical and pharmacological sciences. 2016; 20:2742-51). Topical formulations containing corticosteroids or anesthetics are widely used for relief.

In addition, while combinations of a corticosteroid and lidocaine have been available via pharmaceutical formularies for some time, they have not satisfied the need of this patient population.

There are numerous over the counter (OTC) products used to treat hemorrhoids, and combinations compounded by compounding pharmacies are being used off-label for the treatment of hemorrhoids. There are no prescription products on the market specifically approved by the Food and Drug Administration (FDA) for treating hemorrhoids. There remains a need for a hemorrhoidal treatment option that has been studied in a patient population and that can provide patients a safe, and effective therapy to treat hemorrhoidal disease, Grades I through IV, particularly Grades II and III.

### BRIEF SUMMARY

The following aspects and embodiments thereof described below are meant to be exemplary and illustrative, not limiting in scope.

In one aspect, the disclosure relates to a pharmaceutical composition comprising lidocaine and halobetasol propionate, wherein the lidocaine is present in an amount of about 5% by weight of the total composition and wherein the halobetasol propionate is present in an amount from about 0.02% to about 0.07% by weight of the total composition.

In one aspect, the pharmaceutical composition comprising lidocaine and halobetasol propionate, wherein the lidocaine is present in an amount of about 5% by weight of the total composition, and wherein the halobetasol propionate is present in an amount from about 0.02% to about 0.07% by weight of the total composition.

In one aspect, the lidocaine in the pharmaceutical composition is lidocaine hydrochloride. In such aspects, the lidocaine hydrochloride is present in an amount of about 5.78 wt % (equivalent to about 5 wt % lidocaine).

In other aspects, the halobetasol propionate is present in an amount of about 0.05% by weight of the total composition.

In one aspect, the disclosure relates to a pharmaceutical composition comprising lidocaine and halobetasol propionate that further comprises propylene glycol, an acrylamide sodium/acryloyldimethyl taurate copolymer such as Sepineo® P600, hydroxylpropyl methylcellulose (also known as hypromellose), and purified water.

In one aspect, the pharmaceutical composition comprises propylene glycol that is present in an amount from about 25% to about 35% by weight of the total composition.

In other aspects, the propylene glycol is present in an amount of about 30% by weight of the total composition.

In one aspect, the pharmaceutical composition comprises acrylamide sodium/acryloyldimethyl taurate copolymer that is present in an amount from about 0% to about 10% by weight of the total composition.

In other aspects, the acrylamide sodium/acryloyldimethyl taurate copolymer is present in an amount of about 4% by weight of the total composition.

In one aspect, the pharmaceutical composition comprises hydroxylpropyl methylcellulose that is present in an amount from about 0% to about 5% by weight of the total composition.

In other aspects, the hydroxylpropyl methylcellulose is present in an amount of about 1.25% by weight of the total composition.

In one aspect, the disclosure relates to a pharmaceutical composition comprising lidocaine and halobetasol propionate that further comprises propylene glycol, diethylene glycol monoethylether (DGME/Transcutol-P), glycerin, hydroxylpropyl cellulose, and purified water.

In one aspect, the pharmaceutical composition comprises propylene glycol that is present in an amount from about 25% to about 35% by weight of the total composition.

In other aspects, the propylene glycol is present in an amount of about 1.25% by weight of the total composition.

In one aspect, the pharmaceutical composition comprises diethylene glycol monoethylether/transcutol-P that is present in an amount from about 0% to about 30% by weight of the total composition.

In other aspect, the diethylene glycol monoethylether/transcutol-P is present in an amount of about 20% by weight of the total composition.

In one aspect, the pharmaceutical composition comprises glycerin that is present in an amount from about 5% to about 25% by weight of the total composition.

In other aspects, the glycerin is present in an amount of about 1.25% by weight of the total composition.

In one aspect, the pharmaceutical composition comprises hydroxylpropyl cellulose that is present in an amount from about 0% to about 5% by weight of the total composition.

In other aspects, the hydroxylpropyl cellulose is present in an amount of about 2% by weight of the total composition.

In some aspects, the pharmaceutical composition disclosed herein are stable for at least 4 hours.

In one aspect, the lidocaine in the pharmaceutical composition permeates the stratum corneum (SC) and reaches the epidermis (Epi).

In another aspect, the lidocaine in the pharmaceutical composition permeates the epidermis (Epi) and reaches the dermis (Der).

In one aspect, the halobetasol propionate in the pharmaceutical composition permeates the stratum corneum (SC) and reaches epidermis (Epi).

In another aspect, the halobetasol propionate in the pharmaceutical composition permeates the epidermis (Epi) and reaches the dermis (Der).

The disclosure also relates to a method of treating an anorectal disorder in a subject in need thereof, comprises administering the pharmaceutical compositions disclosed herein.

In such aspects, the anorectal disorder is selected from hemorrhoids, anal warts, anal fissures, anorectal abscesses and anal fistulas.

In some specific aspects, the anorectal disorder is a hemorrhoid.

In one aspect, the hemorrhoid is a Goligher Grade II hemmorrhoid.

In another aspect, the hemorrhoid is a Goligher Grade III hemmorrhoid.

In the methods of the disclosure, the pharmaceutical composition is in the form of a gel or a cream.

In some aspects, the pharmaceutical composition is suitable for topical administration.

In one aspect, the pharmaceutical composition is applied topically to the perianal and lower rectal areas.

In another aspect, the pharmaceutical composition is applied twice daily. The twice daily application may continue in one aspect for up to 1 week, for up to 2 weeks, for up to 8 days or for up to 15 days.

In another aspect, the pharmaceutical composition results in reduction of one or more symptoms in the subject. The one or more symptoms comprise of pain, burning, itching, swelling, and bleeding.

In aspects of this disclosure, the subject is a human patient.

### BRIEF DESCRIPTION OF THE DRAWINGS

FIG. 1A shows the delivery of Halobetasol Propionate to stratum corneum (SC), epidermis (Epi) and dermis (Der) in Formulation 1. FIG. 1B shows the delivery of Halobetasol Propionate to stratum corneum (SC), epidermis (Epi) and dermis (Der) in Formulation 2.

FIG. 2A shows the delivery of Halobetasol Propionate to stratum corneum (SC), epidermis (Epi) and dermis (Der) in Formulation 1. FIG. 2B shows the delivery of Halobetasol Propionate to stratum corneum (SC), epidermis (Epi) and dermis (Der) in Formulation 2.

FIG. 3 shows the delivery of Lidocaine Hydrochloride in the receiver media in Formulations 1 and 2 respectively.

### DETAILED DESCRIPTION

The present disclosure is directed to a pharmaceutical composition comprising a combination of halobetasol propionate (HP) and lidocaine hydrochloride (LH) to treat hemorrhoids. Such compositions are non-occlusive, stable, permeate the epidermis, and exhibit adequate chemical and physical stability to treat hemorrhoids.

In specific aspects, the disclosure relates to a pharmaceutical composition comprising lidocaine and halobetasol propionate, wherein the lidocaine is present in amount about 5% by weight of the total composition and wherein the halobetasol propionate is present in an amount from about 0.02% to about 0.07% by weight of the total composition.

In some aspects, the lidocaine in the pharmaceutical compositions disclosed herein is a salt of lidocaine, such as, but not limited to, lidocaine hydrochloride (LH). In such aspects, LH is present in an amount of about 5.78 wt % (5.78% Lidocaine HCL is equivalent to about 5 wt % lidocaine free base).

### I. Definitions

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Where a range of values is provided, it is intended that each intervening value between the upper and lower limit of that range and any other stated or intervening value in that stated range is encompassed within the disclosure. For example, if a range of 1 mg to 8 mg is stated, it is intended that 2 mg, 3 mg, 4 mg, 5 mg, 6 mg, and 7 mg are also explicitly disclosed, as well as the range of values greater than or equal to 1 mg and the range of values less than or equal to 8 mg.

As used herein, the term “active agent,” “active pharmaceutical ingredient,” or “API” refers to a pharmaceutically active agent or a drug. All these terms also may be used interchangeably. Furthermore, these terms can also refer to halobetasol propionate (HP) and/or lidocaine hydrochloride (LH).

As used herein, the terms “approximately” and “about” refer to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

As used herein, the term “excipient” herein includes any substance used as a vehicle for delivery of the active ingredient to a subject, and any substance added to the active ingredient, for example to improve its handling properties or to permit the resulting composition to be formed into a deliverable unit dose having the desired shape and consistency. The term “excipient” includes inert substances as well as functional excipients that may result in beneficial properties of the composition. Exemplary excipients include but are not limited to polymers, glidants, sugars, lubricants, salts, buffers, fats, fillers, disintegrating agents, binders, surfactants, high surface area substrates, carriers, matrix materials, and so forth.

As used herein, the term “halobetasol propionate (HP)” refers to (6α, 11β, 16β)-21-Chloro-6,9-difluoro-11-hydroxy-16-methyl-17-(1-oxopropoxy) pregna-1,4-diene-3,20-dione or 21-Chloro-6α,9-difluoro-11β,17-dihydroxy-16β-methylpregna-1,4-diene-3,20-dione-17-propionate. HP is a corticosteroid. HP has a molecular weight of 484.96 Da.

The chemical structure of halobetasol propionate is shown below.

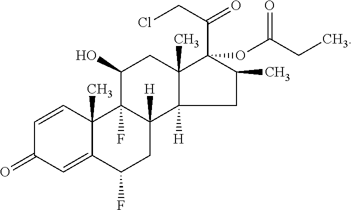

As used herein, “hemorrhoids” refer to swollen varicose veins in the mucous membrane inside or just outside the rectum. The composition and methods of the invention may be used to treat diseases of the anorectum which manifest one or more symptoms of itching, discomfort, pain and bleeding. Accordingly, references to use of the composition and/or methods for treating hemorrhoids is equally applicable to diseases of the anorectum manifesting one or more symptoms in common with hemorrhoids.

As used herein, the term “in vitro” refers to events that occur in an artificial environment, e.g., in a test tube or reaction vessel, in cell culture, in a petri dish, etc., rather than within an organism (e.g., animal, plant, or microbe).

As used herein, the term “in vivo” refers to events that occur within an organism (e.g., animal, plant, or microbe or cell or tissue thereof).

As used herein, the term “lidocaine hydrochloride (LH)” refers to 2-(diethylamino)-N-(2,6-dimethylphenyl) acetamide hydrochloride. LH has a molecular weight of 270.8 Da. LH is an amide local anesthetic.

The chemical structure of Lidocaine HCl is shown below.

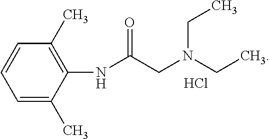 [SMILES: CCN(CC)CC(=O)Nc1c(C)cccc1C ; Cl]

As used herein, the term “mammal” includes humans and both domestic animals such as laboratory animals and household pets (e.g., cats, dogs, swine, cattle, sheep, goats, horses, rabbits), and non-domestic animals such as wildlife and the like.

As used herein, the term “pharmaceutically acceptable” refer to those compounds, salts, compositions, dosage forms, etc., which are—within the scope of sound medical judgment—suitable for use in contact with the tissues of human beings and/or other mammals without excessive toxicity, irritation, allergic response, or other problem or complication, commensurate with a reasonable benefit/risk ratio. In some aspects, “pharmaceutically acceptable” means approved by a regulatory agency of the federal or a state government, or listed in the U.S. Pharmacopeia or other generally recognized pharmacopeia for use in mammals (e.g., animals), and more particularly, in humans.

As used herein, the term “pharmaceutically acceptable carrier” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier that are acceptable for use in humans or domestic animals.

As used herein, the term “pharmaceutical composition” refers to the composition comprising the API along with pharmaceutically acceptable excipients for topical delivery of the API to mammals.

As used herein, the term “subject,” “individual,” or “patient” are used interchangeably and include any animal to which a composition in accordance with the disclosure may be administered. e.g., for experimental, diagnostic, prophylactic, and/or therapeutic purposes. Typical subjects include animals, such as, but not limited to mammals, such as, but not limited to mice, rats, rabbits, non-human primates, and humans.

As used herein, the term “therapeutic agent” refers to any agent that, when administered to a subject, has a therapeutic effect and/or elicits a desired biological and/or pharmacological effect. Therapeutic agents are also referred to as “actives” or “active agents.”

As used herein, the term “effective amount” or “therapeutically effective amount” refers to the amount of the composition which, when administered to a mammal, preferably a human, is sufficient to effect treatment in the mammal, preferably a human. The amount of composition which constitutes a “therapeutically effective amount” will vary depending on the condition and its severity, the manner of administration, and the age of the mammal to be treated, but can be determined routinely by one of ordinary skill in the art having regard to his own knowledge and to this disclosure.

As used herein, the term “treating” or “treatment” as used herein covers the treatment of the disease or condition of interest in a mammal, preferably a human, having the disease or condition of interest, and includes preventing the disease or condition from occurring in a mammal, in particular, when such mammal is predisposed to the condition but has not yet been diagnosed as having it, inhibiting the disease or condition, i.e., arresting its development, relieving the disease or condition, i.e., causing regression of the disease or condition, or relieving the symptoms resulting from the disease or condition, i.e., relieving pain without addressing the underlying disease or condition. The term “treating” also includes any effect, e.g., lessening, reducing, modulating, or eliminating, that results in the improvement of the condition, disease, disorder and the like.

The compositions of the present disclosure can comprise, consist essentially of, or consist of, the components disclosed.

All percentages, parts and ratios are based upon the total weight of the compositions and all measurements made are at about 25° C., unless otherwise specified.

All ranges recited herein include the endpoints, including those that recite a range “between” two values. Term “substantially” and “about” is to be construed as modifying a term or value such that it is not an absolute. This includes, at very least, the degree of expected experimental error, technique error and instrument error for a given technique used to measure a value.

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual excipients, polymers, compounds, or groups thereof, or any members of a claimed group, less than the full measure of this disclosure can be claimed for any reason.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

### II. Pharmaceutical Compositions

In one aspect, provided herein is a pharmaceutical composition comprising lidocaine and halobetasol propionate, wherein the lidocaine is present in amount of about 5% by weight of the total composition and wherein the halobetasol propionate is present in an amount from about 0.02% to about 0.07% by weight of the total composition.

In another aspect, provided herein is a pharmaceutical composition comprising lidocaine and halobetasol propionate, wherein the lidocaine is present in an amount of about 5% by weight of the total composition, wherein the halobetasol propionate is present in an amount from about 0.02% to about 0.07% by weight of the total composition.

In some aspects, the pharmaceutical composition comprise lidocaine present in amount of about 5% by weight of the total composition and halobetasol propionate present in an amount of about 0.05% by weight of the total composition.

In some aspects, the pharmaceutical composition comprise lidocaine present in amount of about 5% by weight of the total composition, and halobetasol propionate present in an amount about of 0.05% by weight of the total composition.

In some aspects, the lidocaine in the pharmaceutical compositions disclosed herein is a salt of lidocaine, such as, but not limited to, lidocaine hydrochloride (LH). In such aspects, LH is present in an amount of about 5.78 wt % (equivalent to about 5 wt % lidocaine).

In some specific aspects, the pharmaceutical composition comprise lidocaine hydrochloride present in amount of about 5.78% by weight of the total composition and halobetasol propionate present in an amount of about 0.02% by weight of the total composition.

In some aspects, the pharmaceutical composition comprise lidocaine hydrochloride present in amount of about 5.78% by weight of the total composition and halobetasol propionate present in an amount of about 0.05% by weight of the total composition.

In some aspects, the pharmaceutical composition comprises lidocaine hydrochloride present in amount of about 5.78% by weight of the total composition, and halobetasol propionate present in an amount of about 0.02% by weight of the total composition.

In some aspects, the pharmaceutical composition comprise lidocaine hydrochloride present in amount of about 5.78% by weight of the total composition, halobetasol propionate present in an amount of about 0.05% by weight of the total composition.

In aspects of the disclosure, the pharmaceutical compositions described herein can further include humectants/solubilizers, bioadhesive agents, gelling agents, co-solvents, diluents, or a combination thereof.

Humectants useful in the practice of the disclosure include, but are not limited to, propylene glycol, glycerin, or mixtures thereof.

In some preferred aspects of the disclosure, the pharmaceutical composition comprising lidocaine hydrochloride and halobetasol propionate includes propylene glycol as a humectant.

In other preferred aspects of the disclosure, the pharmaceutical composition comprising lidocaine hydrochloride and halobetasol propionate includes propylene glycol and glycerin as humectants.

In the aspects of the disclosure, the pharmaceutical composition comprises propylene glycol present in an amount from about 25% to about 35% by weight of the total composition. In specific aspects, the pharmaceutical composition comprises propylene glycol present in an amount of about 30% by weight of the total composition. In other specific aspects, the pharmaceutical composition comprises propylene glycol present in an amount of about 20% by weight of the total composition.

In the aspects of the disclosure, the pharmaceutical composition comprises glycerin present in an amount from about 5% to about 25% by weight of the total composition. In specific aspects, the pharmaceutical composition comprises glycerin present in an amount of about 10% by weight of the total composition.

In specific aspects, the pharmaceutical composition comprises propylene glycol present in an amount of about 20% by weight of the total composition and glycerin present in an amount of about 10% by weight of the total composition.

Bioadhesive agents useful in the practice of the disclosure include, but are not limited to, bioadhesive polymers or mucoadhesive polymers like cellulose derivatives. Such polymers may include, but not limited to hydroxypropyl cellulose (HPC) and hydroxypropyl methylcellulose (HPMC).

In some preferred aspects of the disclosure, the pharmaceutical composition comprising lidocaine hydrochloride and halobetasol propionate includes hydroxypropyl cellulose (HPC).

In other preferred aspects of the disclosure, the pharmaceutical composition comprising lidocaine hydrochloride and halobetasol propionate includes hydroxypropyl methyl cellulose (HPMC).

In the aspects of the disclosure, the pharmaceutical composition comprises hydroxypropyl cellulose (HPC) present in an amount from about 0% to about 5% by weight of the total composition. In specific aspects, the pharmaceutical composition comprises HPC present in an amount of about 2% by weight of the total composition.

In the aspects of the disclosure, the pharmaceutical composition comprises hydroxypropyl methyl cellulose (HPMC) present in an amount from about 0% to about 5% by weight of the total composition. In specific aspects, the pharmaceutical composition comprises HPMC present in an amount of about 1.25% by weight of the total composition.

Gelling agents useful in the practice of the disclosure include, but not limited to acrylamide/sodium acryloyldimethyl taurate copolymer, polysorbate 80, isohexadecane, or a combination thereof.

In some preferred aspects of the disclosure, the pharmaceutical composition comprising lidocaine hydrochloride and halobetasol propionate includes a gelling agent such as a 3 in 1 polymer of acrylamide/sodium acryloyldimethyl taurate copolymer, and polysorbate 80, and isohexadecane, such as Sepineo® P600.

Sepineo® P600 is a thickening, emulsifying and stabilizing polymer useful in pharmaceutical formulations. It is easy to use as it does not require neutralization or rehydration steps when developing creams for topical application.

In the aspects of the disclosure, the pharmaceutical composition comprises the 3 in 1 polymer present in an amount from about 0% to about 10% by weight of the total composition. In specific aspects, the pharmaceutical composition comprises the 3 in 1 polymer present in an amount about 4% by weight of the total composition.

Co-solvents useful in the practice of the disclosure include, but not limited to diethylene glycol monoethylether (DGME/transcutol-P).

In some preferred aspects of the disclosure, the pharmaceutical composition comprising lidocaine hydrochloride and halobetasol propionate includes a co-solvent such as diethylene glycol monoethylether (DGME/transcutol-P).

In the aspects of the disclosure, the pharmaceutical composition comprises the diethylene glycol monoethylether (DGME/transcutol-P) present in an amount from about 0% to about 30% by weight of the total composition. In specific aspects, the pharmaceutical composition comprises the diethylene glycol monoethylether (DGME/transcutol-P) present in an amount about 20% by weight of the total composition.

Diluents useful in the practice of the disclosure include, but not limited to purified water.

In some preferred aspects of the disclosure, the pharmaceutical composition comprising lidocaine hydrochloride and halobetasol propionate includes purified water as the diluent.

In one aspect, provided herein is a pharmaceutical composition comprise lidocaine hydrochloride in amount of about 5.78% by weight of the total composition, halobetasol propionate in amount of about 0.02% by weight of the total composition, propylene glycol, acrylamide sodium/acryloyldimethyl taurate copolymer (and) polysorbate 80 (and) isohexadecane, hydroxylpropyl methylcellulose, and purified water.

In one aspect, provided herein is a pharmaceutical composition comprise lidocaine hydrochloride in amount of about 5.78% by weight of the total composition, halobetasol propionate in amount of about 0.05% by weight of the total composition, propylene glycol, acrylamide sodium/acryloyldimethyl taurate copolymer (and) polysorbate 80 (and) isohexadecane, hydroxylpropyl methylcellulose, and purified water.

In one aspect, provided herein is a pharmaceutical composition comprise lidocaine hydrochloride in amount of about 5.78% by weight of the total composition, halobetasol propionate in amount of about 0.02% by weight of the total composition, propylene glycol, diethylene glycol monoethylether (DGME/transcutol-P), glycerin, hydroxylpropyl cellulose, and purified water.

In one aspect, provided herein is a pharmaceutical composition comprise lidocaine hydrochloride in amount of about 5.78% by weight of the total composition, halobetasol propionate in amount of about 0.05% by weight of the total composition, propylene glycol, diethylene glycol monoethylether (DGME/transcutol-P), glycerin, hydroxylpropyl cellulose, and purified water.

Such pharmaceutical compositions, for example, can be in any dosage form such as, but not limited to cream, gels, liquids, suspensions, emulsions, solution, suspension, ointments. In preferred aspects, the pharmaceutical composition is formulated in the form of a cream or a gel suitable for single administration or multiple administrations of precise dosages.

In some aspects, the pharmaceutical composition is a fixed-dosed combination (FDC) product strength for the topical treatment and symptomatic relief of hemorrhoids. The FDC formulations comprise lidocaine hydrochloride, 5.78 wt % (equivalent to 5 wt % lidocaine) and halobetasol propionate (0.05 wt % or 0.02 wt %).

The combination formulation comprises LH (5.78% w/w) and HP (0.05% w/w) of the total formulation.

The pharmaceutical compositions disclosed herein is intended for topical application of the affected tissue.

In specific aspects, the pharmaceutical composition is applied topically to the perianal and lower rectal areas.

In other aspects, the composition may be applied intrarectally and/or to the anorectal region.

The typical method of use is application of the pharmaceutical composition to the affected area twice daily for up to one week with gentle rubbing.

The methods and pharmaceutical compositions will typically be used in therapy for human subjects, specifically adults. However, they may also be used to treat similar or identical indications in other animal subjects.

### III. Epidemiology of HD

While hemorrhoidal disease is common, there are no recent or exact prevalence data available (Sandler et al. Rethinking what we know about hemorrhoids. Clinical gastroenterology and hepatology: the official clinical practice journal of the American Gastroenterological Association. 2019; 17:8-15), (Mott et al, Diagnosis and treatment options. Am Fam Phys 2018; 97 (3): 172-179). In their paper published in 1990, Johanson and Sonnenberg analyzed epidemiological data from the National Health Interview Survey, the National Hospital Discharge Survey, the National Disease and Therapeutic Index, and the Morbidity Statistics from General Practice from England and Wales (Johanson et al. The prevalence of hemorrhoids and chronic constipation. An epidemiologic study. Gastroenterology. 1990; 98:380-6). They reported that 10 million people in the US complained of hemorrhoids: a prevalence rate of 4.2% at the time of the publication. About 30% of these presented to physicians, and an average of 1.5 million prescriptions were written annually for hemorrhoid preparations. National Ambulatory Medical Care Survey from 2010 indicated that hemorrhoids were the third most frequent outpatient gastrointestinal diagnosis with nearly 4 million annual office and emergency department visits.

Healthcare resource utilization data may be outdated. However, millions of over-the-counter preparations for hemorrhoids are purchased each year; Preparation H (Richmond, VA) alone accounted for $136 million in sales in 2017 (Sandler et al. Rethinking what we know about hemorrhoids. Clinical gastroenterology and hepatology: the official clinical practice journal of the American Gastroenterological Association. 2019; 17:8-15).

In summary, the prevalence of hemorrhoidal disease is not known with precision, with estimates varying from 4% (Johanson et al. 1990) to as high as 39% (Sandler et al. 2019) from colonoscopy data. No credible health care resource utilization studies have been published (Johanson et al. 1990).

The pharmaceutical compositions described herein may also be used in combination with other therapies for treating the same disease. Such combination use includes administration of the pharmaceutical compositions and one or more other therapeutics at different times, or co-administration of the pharmaceutical compositions and one or more other therapies. In some embodiments, dosage may be modified for pharmaceutical compositions of the disclosure or other therapeutics used in combination, e.g., reduction in the amount dosed relative to a pharmaceutical composition or therapy used alone, by methods well known to those of ordinary skill in the art.

It is understood that use in combination includes use with other therapies, drugs, medical procedures etc., where the other therapy or procedure may be administered at different times (e.g. within a short time, such as within hours (e.g. 1, 2, 3, 4-24 hours), or within a longer time (e.g. 1-2 days, 2-4 days, 4-7 days, 1-4 weeks)) than the pharmaceutical compositions described herein, or at the same time as the pharmaceutical compositions described herein.

Provided herein, is an embodiment for a method of treating of an anorectal disorder in a subject comprising administering a pharmaceutical composition of the disclosure.

Also provided herein, in another embodiment, is a use of the pharmaceutical composition described herein for the preparation of medicament for the treatment of an anorectal disorder in a subject.

Also provided herein, is a method of determining whether a pharmaceutical composition comprising lidocaine and halobetasol propionate, wherein the lidocaine is present in an amount of about 5% by weight of the total composition and wherein the halobetasol propionate is present in an amount from about 0.02% to about 0.05% by weight of the total composition is effective in treating a hemorrhoid, the method comprising delivering the pharmaceutical composition to a patient population and assaying the results.

### IV. Hemorrhoidal Disease (HD)

In one aspect, the disclosure provides a pharmaceutical composition for treating an anorectal disorder in a subject comprising lidocaine hydrochloride and halobetasol propionate, wherein the lidocaine hydrochloride is present in amount about 5.78% by weight of the total composition and wherein the halobetasol propionate is present in an amount from about 0.02% to about 0.05% by weight of the total composition.

In aspects of the disclosure, the anorectal disorder is selected from hemorrhoids, anal warts, anal fissures, anorectal abscesses and anal fistulas.

In specific aspects of the disclosure, the anorectal disorder is a hemorrhoid.

In yet other aspect of the disclosure, the hemorrhoid is a Goligher Grade II hemmorhoid.

In other aspect of the disclosure, the hemorrhoid is a Goligher Grade III hemmorhoid.

Hemorrhoids is the swelling of veins in the lower part of the rectum, leading to severe itching, pain, and bleeding. Hemorrhoids are vascular cushions that underlie the distal rectal mucosa and contribute approximately 15-20% of the resting anal pressure, ensuring complete closure of the anal canal. Hemorrhoids become symptomatic when enlarged, inflamed, thrombosed, or prolapsed, and the most common symptoms are painless rectal bleeding, pain, burning, and itching (Gallo G 2018). The most widely used classification system defines four grades of hemorrhoids (Goligher 1980).
Grade I: Normal appearance externally, bleeding but not prolapsing.
Grade II: Anal cushions prolapse on straining but reduces spontaneously.
Grade III: Anal cushions prolapse on straining or exertion and require manual reduction.
Grade IV: Permanent prolapse, irreducible.

### EXAMPLES

The following examples are provided for illustrative purposes only and not to limit the scope of the claims provided herein.

### Example 1: Previous Product Development

A clinical study was previously performed by Applicant using a formulation comprising a combination of hydrocortisone acetate (a corticosteroid) and lidocaine hydrochloride.

A double-blind, vehicle controlled, dose-ranging study was performed. The aim of this study was to evaluate the safety and efficacy of a topical cream with various concentrations of lidocaine hydrochloride and/or hydrocortisone acetate (either alone or in combination) in 211 subjects at nine study centers.

Based on insights derived from the findings of this study, it was decided to increase the potency of the corticosteroid active component, thus, replacing hydrocortisone acetate with halobetasol propionate and change the formulation for delivering the new combination.

### Example 2: Formulation Development

A series of formulations including creams and gels were manufactured and short-term stability was studied.

The cream and gel formulations comprise the APIs, namely, lidocaine hydrochloride, 5.78 wt % (equivalent to 5 wt % lidocaine) and halobetasol propionate (0.05 wt %).

The cream and gel formulations further comprise other additional components.

Some cream formulations comprise purified water as the vehicle or the solubilizer, present in the range from about 36 to about 80% w/w in the total composition.

Some cream formulations comprise propylene glycol as the humectant, present in the range from 0 to about 15% w/w in the total composition.

Some cream formulations comprise transcutol P as the penetration enhancer, present in the range from 0 to about 15% w/w in the total composition.

Some cream formulations comprise isopropyl myristate as the emollient and/or penetration enhancer, present in the range from 0 to about 10% w/w in the total composition.

Some cream formulations comprise oleyl alcohol as the emollient and/or penetration enhancer, and/or solubilizer present in the range from 0 to about 10% w/w in the total composition.

Some cream formulations comprise of medium chain triglycerides as the emollient and/or penetration enhancer, present in the range from 0 to about 10% w/w in the total composition.

Some cream formulations comprise of light mineral oil, present in the range from 0 to about 8% w/w in the total composition.

Some cream formulations comprise formulations can comprise cetyl alcohol, present in the range from 0 to about 7% w/w in the total composition.

Some cream formulations comprise steryl alcohol, present in the range from 0 to about 7% w/w in the total composition.

Some cream formulations comprise glycerol monostearate as the emulsifier, present in the range from 0 to about 4% w/w in the total composition.

Some cream formulations comprise sorbitan monostearate as the surfactant, present in the range from 0 to about 2% w/w in the total composition.

Some cream formulations comprise polysorbate 80 as the surfactant, present in the range from 0 to about 8% w/w in the total composition.

Some cream formulations comprise steareth 20 as the surfactant, present in the range from 0 to about 3% w/w in the total composition.

Some cream formulations comprise Tefose® 63 as the bodifying agent, present in the range from 0 to about 8% w/w in the total composition.

Some cream formulations comprise white ceresin wax as the bodifying agent, present in the range from 0 to about 7% w/w in the total composition.

Some cream formulations comprise PEG 1500 as the humectant and/or bodifying agent, present in the range from 0 to about 10% w/w in the total composition.

Some cream formulations comprise glycerin as the humectant, present in the range from 0 to about 5% w/w in the total composition.

Some cream formulations comprise Sepineo® P600 as the gelling agent, present in the range from 0 to about 4% w/w in the total composition.

Some cream formulations comprise carbopol 974 (homopolymer type B) as the gelling agent, present in the range from 0 to about 1% w/w in the total composition.

Some cream formulations comprise carbopol 980 (homopolymer type C) as the gelling agent, present in the range from 0 to about 0.8% w/w in the total composition.

Some cream formulations comprise hydroxypropyl methyl cellulose as the gelling agent, present in the range from about 0.8 to about 1% w/w in the total composition.

Some cream formulations comprise purified water, propylene glycol, transcutol P, glycerin, Sepineo® P600, hydroxypropyl cellulose, hydroxypropyl methyl cellulose, or a combination thereof.

Some cream formulations comprise purified water as the vehicle or the solubilizer, present in the range from about 42 to about 59% w/w in the total composition.

Some cream formulations comprise propylene glycol as the humectant, present in the range from 20 to about 30% w/w in the total composition.

Some cream formulations comprise transcutol P as the penetration enhancer, present in the range from 0 to about 20% w/w in the total composition.

Some cream formulations comprise glycerin as the humectant, present in the range from 0 to about 10% w/w in the total composition.

Some cream formulations comprise Sepineo® P600 as the gelling agent, present in the range from 0 to about 4% w/w in the total composition.

Some cream formulations comprise hydroxypropyl cellulose as the gelling agent, present in the range from 0 to about 2% w/w in the total composition.

Some cream formulations comprise hydroxypropyl methyl cellulose as the gelling agent, present in the range from 0 to about 1.25% w/w in the total composition.

### 2.1 Gel Formulations

Gel formulations were manufactured utilizing Sepineo® P600 (acrylamide/sodium acryloyldimethyl taurate copolymer/Isohexadecane & polysorbate 80) (Formulation 1).

Gel formulations were also manufactured utilizing diethylene glycol monoethylether (DGME/Transcutol-P) (Formulation 2).

Such gel formulations were also studied. Other viscosity enhancing agents were also included in these formulations.

Hydroxypropyl methylcellulose (HPMC) at 1.25% w/w was identified as optimized concentration to achieve the desired viscosity to the gel formulation.

Hydroxypropyl cellulose (HPC) was investigated as an alternate strategy towards the development of a gel formulation. Low viscosity HPC grade at 2.0% w/w was identified as optimized concentration with propylene glycol (PG) and transcutol p (TP) as permeation enhancers and solvents to dissolve both the API's. Since the HP has higher solubility in both the solvents (PG and TP), this formulation resulted in clear and transparent gel with both the APIs solubilized.

### 2.2 Short-Term Stability

All formulations were placed on short term stability. Samples were placed at 25° C./60% RH and 40° C./75% RH and evaluated at T0, T=2 weeks, and T=4 weeks. The appearance and pH of the prototype formulations on stability were observed.

Most of the cream and gel formulations were physically stable. However, some of the cream formulations exhibited phase separation. The formulations either re-dispersed upon gentle shaking or formed lumps upon gentle shaking and hence were not stable.

The pH of all the cream and gel formulations were comparable with no significant difference from T0 to T=4 weeks, except that the pH could not be measured in some of the cream formulations that demonstrated phase separation.

2.3 Solubilized HP vs. Suspended HP

It is well known that solubilized form of API tends to permeate well across the biological tissue. But in some cases, solubilized API tend to have higher affinity towards the vehicle in the formulation and thereby, the release of API from the formulation will be hindered. This could result in decrease permeation across the tissue, despite the API being solubilized. On the contrary, API suspended in the formulation is readily available for absorption, but at the same time, it poses the risk of homogeneity.

In certain aspects, halobetasol proprionate was either suspended or solubilized in the cream and gel formulations.

Some of the cream and gel formulations that comprised suspended HP showed birefringence under polarizer: for example, Formulation 1.

Some of the cream and gel formulations that comprised solubilized HP did not show any birefringence under polarizer: for example, Formulation 2.

### Example 3: Pharmaceutical Formulations

Based on the stability data, the effect of HP in solubilized vs. suspended and the effect of bio-adhesive polymers, the following gel formulations disclosed in Tables 1-2 were prepared and tested towards in vitro delivery of the APIs to the skin.

For each of the tables below, the function shown is the presumed function. Applicant provides this information without being bound by theory. Applicant has not tested each of these functions for all formulations of these examples.

### 3.1 Gel Formulation 1

Formulation 1 was prepared as described above.

TABLE 1
Formulation 1-Halobetasol Suspension Formulation.

| | | | Amount |
|---|---|---|---|
| S. No. | Ingredients | Function | % w/w |
| 1 | Lidocaine HCl | Active | 5.78 |
| 2 | Halobetasol Propionate | Active | 0.05 |
| 3 | Propylene glycol | Humectant/ | 30.0 |
| | | Solubilizer | |
| 4 | Acrylamide/Sodium | Gelling agent | 4.0 |
| | Acryloyldimethyl Taurate | | |
| | Copolymer | | |
| 5 | Polysorbate 80 | | |
| 6 | Isohexadecane | | |
| 7 | Hydroxypropyl methylcellulose | Bioadhesive agent | 1.25 |
| 8 | Purified water | Diluent | Q.s to |
| | | | 100 |

| | |
|---|---|
| Total= | 100 |

### 3.2 Gel Formulation 2

Formulation 2 was prepared as described above.

TABLE 2
Formulation 2-Halobetasol Solubilized Formulation.

| | | | Amount |
|---|---|---|---|
| S. No. | Ingredients | Function | % w/w |
| 1 | Lidocaine HCl | Active | 5.78 |
| 2 | Halobetasol Propionate | Active | 0.05 |
| 3 | Propylene glycol | Humectant/ | 20.0 |
| | | Solubilizer | |
| 4 | Diethylene glycol monoethyl- | Co-Solvent | 20.0 |
| | ether (DGME/ Transcutol-P) | | |
| 5 | Glycerin | Humectant | 10.0 |
| 6 | Hydroxypropyl Cellulose | Bioadhesive agent | 2.0 |
| 7 | Purified water | Diluent | Q.s to 100 |

| | |
|---|---|
| Total= | 100 |

### Example 4: In Vitro Delivery of Halobetasol Propionate and Lidocaine HCl

The ability of the formulations of the present invention to deliver the APIs to their active sites was investigated by in vitro permeation tests.

### 4.1 Delivery of Halobetasol Propionate to the Skin

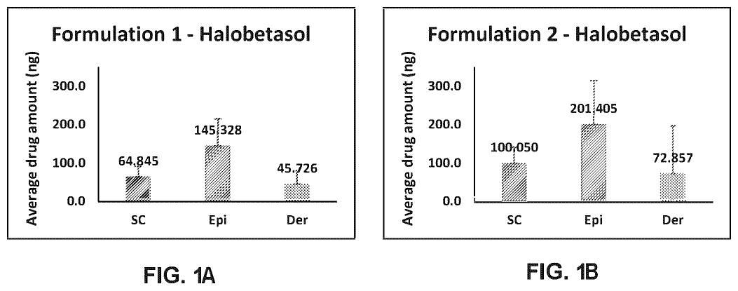

FIG. 1 shows the delivery of Halobetasol Propionate to stratum corneum (SC), epidermis (Epi) and dermis (Der) in Formulations 1 and 2 respectively. FIG. 1A shows the delivery of Halobetasol Propionate to stratum corneum (SC), epidermis (Epi) and dermis (Der) in Formulation 1. FIG. 1B shows the delivery of Halobetasol Propionate to stratum corneum (SC), epidermis (Epi) and dermis (Der) in Formulation 2.

Data from FIG. 1 suggest that Halobetasol Propionate is a highly potent steroid with no receiver media concentration and minimum systemic exposure. Partitioning of Halobetasol from SC to epidermis was higher for Formulation 1 (64 ng to 145 ng). Formulation 2 (˜8%) demonstrated higher % delivery compared to Formulation 1 (˜5.6%).

### 4.2 Delivery of Lidocaine HCl to the Skin

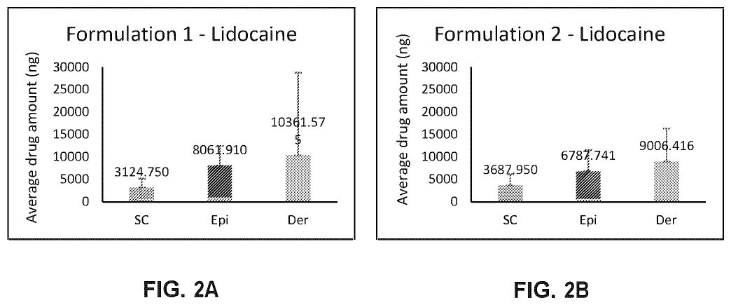

FIG. 2 shows the delivery of Lidocaine Hydrochloride to stratum corneum (SC), epidermis (Epi) and dermis (Der) in Formulations 1 and 2 respectively. FIG. 2A shows the delivery of Halobetasol Propionate to stratum corneum (SC), epidermis (Epi) and dermis (Der) in Formulation 1. FIG. 2B shows the delivery of Halobetasol Propionate to stratum corneum (SC), epidermis (Epi) and dermis (Der) in Formulation 2.

Data from FIG. 2 suggest that the amount of LH concentrations increased from SC to epidermis was higher with Formulation 1 when compared to Formulation 2.

### 4.3 Delivery of Lidocaine HCl in Receiver Media

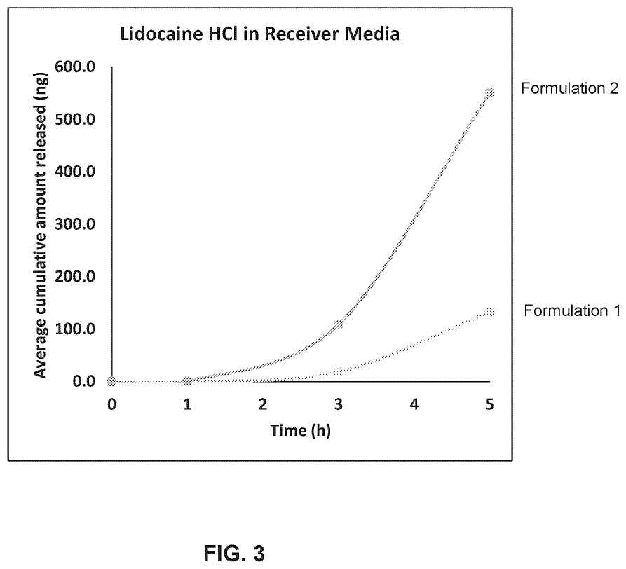

FIG. 3 shows the delivery of Lidocaine Hydrochloride in the receiver media in Formulations 1 and 2 respectively.

Data from FIG. 3 suggest the amount of LH released in the initial hours plays a crucial role, since the local anesthetic action of LH is expected during the early phase of formulation application. When comparing the cumulative amount released into the receiver chamber for the two formulations during first 5 hr, Formulation 1 demonstrated higher lag time when compared to Formulation 2.

### 4.4 Summary of In Vitro Delivery

Results from FIGS. 1-3 indicate that Formulation 2 performed better than Formulation 1 based on the higher % drug delivery into the skin for Halobetasol and noticeable concentrations of Lidocaine in the receiver chamber at 3 h.

Based on the sustain release profile of Lidocaine and good partitioning of Halobetasol from SC to epidermis, Formulation 1 performed better than Formulation 2.

### Example 5: Description of Cream Dosage Form

CITI-002 (Lidocaine HCl/Halobetasol Propionate) cream was manufactured in a fixed-dose combination product strength. Additionally, each active component of CITI-002 was manufactured as individual monads as well as a corresponding placebo. A description of the various drug products is provided in Table 3.

TABLE 3
Description of Creams.

| Contents (8 g tube/cream) | Description |
|---|---|
| Lidocaine Hydrochloride 5.78% w/w/ | CITI-002 combination |
| Halobetasol Propionate 0.05% w/w Cream | cream |
| Lidocaine Hydrochloride 5.78% w/w/ | Lidocaine HCl |
| Halobetasol Propionate Free Cream | monad cream |
| Lidocaine Hydrochloride Free/Halobetasol | Halobetasol propionate |
| Propionate 0.05% w/w Cream | monad cream |
| Lidocaine Hydrochloride Free/Halobetasol | Placebo cream |
| Propionate Free Cream | |

### 5.1 Composition

The unit composition of the various drug product creams is presented in Table 4.

TABLE 4
Unit Composition, Pharmaceutical Function
Quality Standards and Ingredients.

| | | CITI- | | | | Quality |
|---|---|---|---|---|---|---|
| | | 002 LH | LH | | | Stan- |
| Raw | | & HP | Monad | HP | Placebo | dards |
| Material | Function | % w/w | % w/w | % w/w | % w/w | % w/w |
| Lidocaine | Drug | 5.78 | 5.78 | — | — | USP/NF |
| HCl (LH) | substance | | | | | |
| Halobetasol | Drug | 0.05 | — | 0.05 | — | USP/NF |
| Propionate | substance | | | | | |
| (HP) | | | | | | |
| Purified | Water | 58.92 | 58.97 | 64.70 | 64.75 | USP |
| Water | | | | | | |
| Propylene | Pene- | 30.00 | 30.00 | 30.00 | 30.00 | USP |
| Glycol | tration | | | | | |
| | enhancer | | | | | |
| Sepineo | Gelling | 4.00 | 4.00 | 4.00 | 4.00 | N/A |
| P600 ® | agent | | | | | |
| Hydroxy | Lubricant | 1.25 | 1.25 | 1.25 | 1.25 | USP/NF |
| propyl | | | | | | |
| methyl | | | | | | |
| cellulose | | | | | | |
| | Total | 100.00 | 100.00 | 100.00 | 100.00 | |

5.78% lidocaine HCl is equivalent to 5% lidocaine free base.
Purified water concentration = Q.S., water is added to achieve a total drug product concentration of 100%.

5.78% lidocaine HCl is equivalent to 5% lidocaine free base.
Purified water concentration=Q.S., water is added to achieve a total drug product concentration of 100%.

### Example 6: In Vivo Delivery of Halobetasol Propionate and Lidocaine HCl

The efficacy and safety of delivering the pharmaceutical composition comprising Lidocaine HCl and Halobetasol Propionate (CITI-002) towards topical treatment and symptomatic relief of hemorrhoids was studied on a patient population in a clinical trial. As discussed below, the greatest efficacy was observed with CITI-002, where 42% of subjects achieved success from Day 8 to end of treatment. CITI-002 was demonstrated to be more effective than its individual components.

This trial is a multi-center, randomized, double-blind, parallel-group comparison of CITI-002 versus its monads. Qualifying subjects presenting with Grade II or III hemorrhoids were randomized to treatment with one of five test articles (1:1 for all groups) applied twice daily to the peri-rectal area as well as the distal aspect of the anal canal using the product applicator tip.

The treatment arms were as follows:
A single therapy cream containing a topical anesthetic, Lidocaine HCl, 5.78%.
A single therapy cream containing a topical corticosteroid, Halobetasol Propionate (0.05%).
A combination cream containing Lidocaine HCl and Halobetasol Propionate (5.78%/0.02%)-referred as CITI-002 Low.
A combination cream containing Lidocaine HCl and Halobetasol Propionate (5.78%/0.05%)-CITI-002, also referred as CITI-002 High.

For clarity, reference to CITI-002 in this example refers to CITI-002 High unless otherwise specified.

### 6.1 Hemorrhoid Quality of Life Index (HQLI)

This Example utilizes the exemplary Hemorrhoid Quality of Life Index (HQLI) as an indicator to evaluate the effectiveness of CITI-002 in treating Grades II and III hemorrhoids in the clinical trial. The exemplary HQLI includes two parts.

Part I comprises a set of five hemorrhoidal disease symptom intensity items intended to be assessed daily with a 24-hour recall period. This assessment employs a 11-point numerical rating scale (NRS) with anchors at 0 for no (symptom), 5 for moderate (symptom), and 10 for worst possible (symptom).

Part II includes ten items that incorporate a 7-day recall period for weekly administration. These items utilize five-category verbal response scales ranging from not at all [impact] to extremely or a great deal [impact].

### 6.2 Efficacy Results

### 6.2.1 Reduction in Symptoms

As shown in Tables 5-9, CITI-002 had a greater treatment effect at Day 8 compared to the component monad treatments for SIS (Table 5), Pain (Table 6), Burning (Table 7), Swelling (Table 8), and Bleeding (Table 9). This greater treatment effect at the end of the treatment across these four individual symptoms, as well as the Symptom Intensity Summary (SIS) Score, shows that the formulation has a greater effect than its component monad ingredients in the modified intention-to-treat (mITT) population.

TABLE 5
SIS Success by MCT
(Reduction ≥ 5.247 points) at Day 8 (mITT).

| | Symptom Intensity |
|---|---|
| Treatment Arm | Summary (SIS) (%) |
| CITI-002 | 42 |
| HBP 0.05% | 29 |
| Lidocaine | 21 |

TABLE 6
Pain Success by MCT
(Reduction ≥ 6 points) at Day 8 (mITT).

| | Symptom Intensity |
|---|---|
| Treatment Arm | Summary (SIS) (%) |
| CITI-002 | 42 |
| HBP 0.05% | 31 |
| Lidocaine | 31 |

TABLE 7
Burning Success by MCT
(Reduction ≥ 6 points) at Day 8 (mITT).

| | Symptom Intensity |
|---|---|
| Treatment Arm | Summary (SIS) (%) |
| CITI-002 | 42 |
| HBP 0.05% | 39 |
| Lidocaine | 30 |

TABLE 8
Swelling Success by MCT
(Reduction ≥ 5 points) at Day 8 (mITT).

| | Symptom Intensity |
|---|---|
| Treatment Arm | Summary (SIS) (%) |
| CITI-002 High | 48 |
| HBP 0.05% | 42 |
| Lidocaine | 39 |

TABLE 9
Bleeding Success by MCT
(Reduction ≥ 3 points) at Day 8 (mITT).

| | Symptom Intensity |
|---|---|
| Treatment Arm | Summary (SIS) (%) |
| CITI-002 | 50 |
| HBP 0.05% | 48 |
| Lidocaine | 44 |

Moreover, proportionally more patients in the CITI-002 cohort reported meaningful and statistically significant improvement as compared to patients treated with lidocaine alone (CMH test, p=0.035).

An assessment of clinical treatment efficacy outcomes during 7-day treatment and 7-day follow-up periods was conducted using an analysis of covariance, which analyzed changes from baseline. Substantial improvements were seen across all active treatment groups. Although no statistical significance was determined in the changes between the comparison groups, directionally the data signaled that the combination products provided faster relief compared to individual monads, and the relief persisted after completing treatment.

### 6.3 Safety Results

There were no serious adverse events.

While the invention has been described by discussion of embodiments of the invention and non-limiting examples thereof, one of ordinary skill in the art may, upon reading the specification and claims, envision other embodiments and variations which are also within the intended scope of the invention and therefore the scope of the invention shall only be construed and defined by the scope of the appended claims.

### EQUIVALENTS

It is to be understood that while the present disclosure has been described in conjunction with the detailed description thereof, the foregoing description is intended to illustrate and not limit the scope of the present disclosure, which is defined by the scope of the appended claims. Other aspects, advantages, and alterations are within the scope of the following claims.

What is claimed is:

1. A pharmaceutical composition comprising lidocaine and halobetasol propionate, wherein the lidocaine is present in an amount of about 5% by weight of the total composition and wherein the halobetasol propionate is present in an amount from about 0.02% to about 0.07% by weight of the total composition.

2. The pharmaceutical composition of claim 1, wherein the lidocaine is lidocaine hydrochloride.

3. The pharmaceutical composition of claim 1, wherein the halobetasol propionate is present in an amount of about 0.05% by weight of the total composition.

4. The pharmaceutical composition of claim 1, further comprises propylene glycol.

5. The pharmaceutical composition of claim 1, further comprising an acrylamide sodium/acryloyldimethyl taurate copolymer such as Sepineo® P600.

6. The pharmaceutical composition of claim 1, further comprising hydroxylpropyl methylcellulose.

7. The pharmaceutical composition of claim 1, further comprising purified water.

8. The pharmaceutical composition of claim 4, wherein the propylene glycol is present in an amount from about 25% to about 35% by weight of the total composition.

9. The pharmaceutical composition of claim 8, wherein the propylene glycol is present in an amount of about 30% by weight of the total composition.

10. The pharmaceutical composition of claim 5, wherein the acrylamide sodium/acryloyldimethyl taurate copolymer is present in an amount from about 0% to about 10% by weight of the total composition.

11. The pharmaceutical composition of claim 10, wherein the acrylamide sodium/acryloyldimethyl taurate copolymer is present in an amount of about 4% by weight of the total composition.

12. The pharmaceutical composition of claim 6, wherein the hydroxylpropyl methylcellulose is present in an amount from about 0% to about 5% by weight of the total composition.

13. The pharmaceutical composition of claim 12, wherein the hydroxylpropyl methylcellulose is present in an amount of about 1.25% by weight of the total composition.

14. The pharmaceutical composition of claim 1, further comprising propylene glycol, diethylene glycol monoethylether (DGME/Transcutol-P), glycerin, hydroxylpropyl cellulose, and purified water.

15. The pharmaceutical composition of claim 14, wherein the propylene glycol is present in an amount from about 25% to about 35% by weight of the total composition.

16. The pharmaceutical composition of claim 1, wherein the lidocaine permeates the stratum corneum and reaches the epidermis.

17. The pharmaceutical composition of claim 1, wherein the lidocaine permeates the epidermis and reaches the dermis.

18. The pharmaceutical composition of claim 1, wherein the halobetasol propionate permeates the stratum corneum and reaches the epidermis.

19. The pharmaceutical composition of claim 1, wherein the halobetasol propionate permeates the epidermis and reaches the dermis.

20. A method of treating an anorectal disorder in a subject thereof, comprises administering a pharmaceutical composition of claim 1.
